(19) United States
(12) Patent Application Publication
(10) Pub. No.: US 2012/0264232 A1
(43) Pub. Date: Oct. 18, 2012

(54) ANTIGEN DETECTION SYSTEM AND METHODS OF USE

(75) Inventors: Ellen R. Kramer, Menlo Park, CA (US); Mitchell Portnoy, Menlo Park, CA (US)

(73) Assignee: Aliment Health, Inc., Menlo Park, CA (US)

(21) Appl. No.: 13/448,354

(22) Filed: Apr. 16, 2012

Related U.S. Application Data
(60) Provisional application No. 61/476,185, filed on Apr. 15, 2011.

(51) Int. Cl. G01N 33/53; G01N 33/561; G01N 27/62; G01N 21/55; G01N 21/41

(52) U.S. Cl. 436/501; 422/69

(57) ABSTRACT

A system and method for testing for the presence of antigens in food stuffs permits a user to test food products for the presence of antigens for a given food allergy that the user may have. The system comprises two main components, a base station and a test well. The user places a sample of food into the test well. A macerator homogenizes the food in the test well. Antibodies to a particular antigen are bound to an antigen detector in the test well. The base station includes a cartridge dock which powers the macerator and the antigen detector. Antigen-antibody binding provides a change detectable by the detector, which signals the base station of the presence of a threshold degree of antigen-antibody binding and alerts the user of the presence of the antigen, such as by a visual or audible indicator.

### CROSS-REFERENCE TO RELATED APPLICATIONS

This application claims priority to U.S. Provisional Patent Application 61/476,185 filed Apr. 15, 2011, which is hereby incorporated in its entirety by reference.

### BACKGROUND OF THE INVENTION

The present invention relates to a system for detecting a target antigen in a specimen sample. Specifically, the invention relates to the detection of allergens or other substances that can cause a allergic response in a subject, such as a human.

Food allergies are a common ailment suffered by individuals. Allergies to substances such as nut oils, gluten, lactose, and shellfish oils can provoke serious physiological responses including dermatitis, gastrointestinal distress, and respiratory distress, including anaphylaxis. In some circumstances, the allergic responses can be so severe as to threaten the life of the individual and require immediate emergency treatment. One such treatment is injecting epinephrine into the individual, which counteracts the vasodilatation caused by anaphylaxis.

Food allergy prevalence in the United States is summarized in Table 1, below.

TABLE 1

| | Percentage of the Population | | | | | | | | |
|---|---|---|---|---|---|---|---|---|---|
| | All | | | | | | | | |
| Age Group | Allergens | Milk | Egg | Peanut | Tree nuts | Fish | Shellfish^{a} | Wheat | Soy |
| Children | 6.0 | 2.5 | 1.3 | 0.8 | 0.2 | 0.1 | 0.0 | UNK^{b} | 0.2 |
| Adults | 3.7 | 0.3 | 0.2 | 0.6 | 0.5 | 0.4 | 2.0 | UNK^{b} | UNK^{b} |

^{a}Shellfish includes both crustaceans and mollusks.
^{b}UNK = unknown.

The United States Centers for Disease Control and Prevention has reported that from 1998-2000 to 2007-2009, the percentage of children who were reported to have a food allergy during the preceding 12 months increased from 3.5% to 4.6%. (United States Department of Health and Human Services, Centers for Disease Control and Prevention, National Center for Health Statistics, Vital and Health Statistics, Series 10, Number 247, Summary Health Statistics for U.S. Children: National Health Interview Survey: 2009) During this same period, hospital discharges in the United States with a diagnosis of food allergy increased from an average of 2,615 per year in 1998-2000 to 9,537 per year for the period of 2004-2006. (United States Department of Health and Human Services, Centers for Disease Control and Prevention, National Center for Health Statistics, NCHS Data Brief No. 10, October, 2008). Thus, not only are food allergies a problem that affects a significant percentage of the population, but it is a problem that results in a large number of hospital visits annually.

Exposure to allergens is often by the consumption of food or beverage containing the harmful substance. For example, when dining in a restaurant, an individual with a food allergy may be unaware that a dish could contain the allergen. In some cases, the dish could be purposefully made with an ingredient containing the substance. In other instances, the dish may be prepared with the same cooking utensils that had previously been used to prepare another dish containing the allergen, thereby transferring the allergen to the dish. In yet further instances, an ingredient in the dish may have been handled or prepared in a facility in which allergen-containing ingredients were also handled or prepared, causing amounts of the allergen sufficient to cause an allergic reaction to be deposited onto the ingredient used in the dish.

Both whole foods and food ingredients may contain food allergens. The United States Food Allergen Labeling and Consumer Protection Act of 2004 (P.L. 108-282) (FALCPA) identifies eight major foods or food groups: milk, eggs, fish (e.g., bass, flounder, cod), crustacean shellfish (e.g., shrimp, crab, lobster), tree nuts (e.g., almonds, walnuts, pecans), peanuts, wheat, and soybeans. These eight foods are believed to account for 90 percent of food allergies and most serious reactions to foods (FALCPA section 202(2)(A); More than 160 other foods are known to cause food allergies; however, these allergies are relatively rare with prevalence rates ranging from a few percent of the allergic population to single cases. Each of the eight major food allergens contains multiple allergenic proteins, many of which have not been fully characterized.

Some food ingredients such as edible oils, hydrolyzed proteins, lecithin, gelatin, starch, lactose, flavors, and incidental additives (e.g., processing aids), may be derived from major food allergens. The role that these ingredients play in food allergy has not been fully characterized. For example, lecithin is a common food ingredient which is often derived from soybeans. It is possible that soy lecithin, which contains residual protein, could elicit an allergic reaction in sensitive individuals. Another example is protein hydrolysate, which is often made from major food allergens such as soybeans, wheat, peanuts, or milk protein. Partially hydrolyzed protein ingredients can elicit allergic reaction. For example, hot dogs formulated with partially hydrolyzed casein have elicited allergic reactions in children allergic to cow's milk. Allergic reactions to partially hydrolyzed protein ingredients are more common than are reactions to extensively hydrolyzed protein ingredients.

Gelatins are ingredients derived from animals (e.g., cows, pigs) but also from the skin of various species of fish. A study of 10 fish allergic patients and 15 atopic individuals with eczema revealed that 3 and 5 individuals respectively had specific IgE to fish gelatin, suggesting the presence of allergenic protein. Sakaguchi, M., et al., (2000) IgE antibody to fish gelatin (type I collagen) in patients with fish allergy. J Allergy Clin Immunol, 106(3):579-584. However, in a recent double-blind placebo-controlled food challenge (DBPCFC) study, all 30 fish allergic subjects in the study showed no response to a cumulative dose of 3.61 g of fish gelatin. Hansen, T., et al., K. (2004), A randomized, double-blinded, placebo-controlled oral challenge study to evaluate the allergenicity of commercial, food-grade fish gelatin. Food Chem Toxicol, 42(12):2037-2044.

Edible oils can be derived from major food allergens such as soybeans and peanuts, and they may contain variable levels of protein. The consumption of highly refined oils derived from major food allergens by allergic individuals does not appear to be associated with allergic reactions. For example, Taylor, S. L., et al., Peanut oil is not allergenic to peanut-sensitive individuals. J Allergy Clin Immunol, 68(5):372-375 (1981) and Bush, R. K., et al., Soybean oil is not allergenic to soybean-sensitive individuals. J Allergy Clin Immunol, 76(2 Pt 1):242-245 (1985) did not observe any reactions to refined peanut or soy oils in 10 and 7 allergic patients, respectively. On the other hand, unrefined or cold-pressed oils that contain higher levels of protein residues may cause allergic reactions. For example, it has been reported that 6 of 60 peanut allergic individuals reacted to crude peanut oil but none responded to refined peanut oil. Similarly, it has been reported that 15 of 41 peanut allergic children responded positively to crude peanut oil in skin prick tests, but none responded to refined peanut oil. The actual protein levels reported in various edible oils varies, probably due to differences in the oil, refining process, and the protein detection analytical method used. Crude peanut and sunflower oils contained 100 to 300 μg/ml of protein, but that the most highly refined oils contained 0.2 to 2.2 μg/ml of protein. Intermediate protein concentrations were seen for partially processed oils. Teuber et al. (1997) showed that the amount of protein in both crude and refined gourmet nut oils varied both by type of oil and degree of processing; the reported values ranged from 10 to 60 μg/ml for various unrefined oils and from 3 to 6 μg/ml for the refined oils. Other investigators reported undetectable levels of proteins in refined edible oils using assays with detection sensitivities of <0.3 ng/ml and 0.4 mg/kg.

Allergens, or proteins derived from allergenic foods, may be present in foods as the result of cross-contact during processing and handling. The term “cross-contact” describes the inadvertent introduction of an allergen into a product that would not intentionally contain that allergen as an ingredient. Cross-contact may occur when a residue or other trace amount of a food allergen is present on food contact surfaces, production machinery, or is air-borne, and unintentionally becomes incorporated into a product not intended to contain, and not labeled as containing, the allergen. Cross-contact may also result when multiple foods are produced in the same facility or on the same processing line, through the misuse of rework, as the result of ineffective cleaning, or may result from customary methods of growing and harvesting crops, as well as from the use of shared storage, transportation, or production equipment. Cross-contact of foods with allergens has been shown to lead to allergic reactions in consumers on numerous occasions. Much cross-contact can be avoided by controlling the production environment.

In order to avoid an allergic reaction, allergy-prone individuals need a way to rapidly and readily determine in advance if a food product they are contemplating consuming contains a particular antigen. This problem is particularly acute where an allergic individual is ordering prepared foods, such as at a restaurant. Presently, no options exist to allow allergic individuals to rapidly and accurately test prepared food products for the presence of antigens to which they may have a reaction. Knowledge of an allergen's presence is restricted to questioning the restaurant staff and perhaps examining food labels. Where the waiter or food preparer happens to know the allergen is present, e.g., where peanut oil is used in preparing the dish, or the dish is made with gluten, the individual can avoid the dish and thereby avoid an allergic reaction. However, when neither the waiter nor the food preparer know if the allergen is present or not, the individual consumer no objective information to rely upon to insure that the dish does not contain the allergen. Typically, this process is highly risky any individual with a food allergy.

A first diagnostic test known in the art is a lateral flow immunochromatographic assay (LFI). LFI tests are frequently used in home pregnancy test kits. An example of a common implementation of an LFI is the Neogen® Reveal® series of products (http://www.neogen.com/FoodSafety/R_Index.html). Typically, LFI tests come in dipstick form, where one end of a test strip is coated with the sample. Through capillary action, the sample flows along the test strip, encountering both a coloring agent as well as antibodies selected to bind to a target allergen. If the sample does contain the target allergen, the antibodies deposited on the test strip will bind to the coloring agent-treated allergen, causing a change in the surface plasmon resonance of the coloring agent. As a result, the coloring agent will make an observable change in color, indicating the presence of the target allergen.

One disadvantage of LFI tests is the length of time for the capillary action to cause the sample to flow through the test strip. As with the Neogen® Reveal® LFIs, a testing time of ten minutes is common.

Another disadvantage of common implementations of LFI tests for allergens is that they require significant sample and reagent preparation in order to ensure that the sample is as homogenously mixed as possible. Testing of pre-prepared foods is generally not feasible with LFI tests, as one cannot simply dip the test stick into the prepared food and obtain an accurate reading. Moreover, even within the sample, it is inherent in the nature of LFI tests to only test the sample that coats and is absorbed by the end of the dipstick that gets coated. If the amount of the sample absorbed by the test strip happens to contain a lower amount of the allergen than is present in the rest of the object, than the test results may not properly indicate the presence or absence of the allergen.

Other diagnostic tests are known in the industry. For instance, the Biacore™ 4000 is a laboratory-grade device for detecting a target allergen in a sample (http://www.biacore.com/lifesciences/products/systems_overview/Biacore_{—}4000/Antibody-Analysis/index.html). However, this device is both too expensive for individual consumer use and is not sufficiently portable to be used outside of a laboratory.

Aside from diagnostic tests, which are neither readily available to consumers or practical for use by an individual consumer with a food allergy, one could estimate the risk for allergens by consulting information provided by previous diners. There are many internet websites that allow diners to post comments about specific restaurants, and those comments could include that user's experience regarding their own food allergies. Additionally, websites such as www.lonelyplate.org are dedicated solely to diner reviews regarding their experiences with individual restaurants and food allergens. However, all of these types of websites rely on the previous diners to either consume the food and risk an allergic reaction to determine the presence or absence of allergens in the food they order, and then communicate, perhaps incorrectly or inaccurately, what they learned on the website by manually inputting their own description. Currently, there is not an option for electronically conveying the results of an allergen diagnostic test in an automatic fashion over a network, such as the internet.

Accordingly, there is a need for a system to objectively detect allergens in samples taken from food and to do so virtually immediately to provide substantially real-time information to the consumer concerning the presence of absence of an objectionable antigen in the food the consumer may consume. Moreover, there is a need for a system to provide increase the homogenization of samples to increase the efficacy of food antigen detection. Additionally, the system must be sufficiently portable to be carried by an individual consumer and used in the field or in day-to-day life. Finally, there is a need for a system capable of electronically delivering the results of an allergen diagnostic test automatically over a network, such as the internet.

### SUMMARY OF THE INVENTION

In accordance with the present invention, a portable system for detecting the presence of antigens in a food sample and a method of use thereof are disclosed. One aspect of the invention is the use of a macerator, synonymously termed throughout the application as a homogenizer, to break down the food sample and increase the even distribution of antigens in the sample. Another aspect of the invention is the use of a battery to increase the portability of the system.

In at least one embodiment, the antigen detection system includes a sample test well, which may be housed in a cartridge, a docking station or other type of recepticle. The sample test well can include a chamber for disposition of the sample, a homogenizing mechanism, and an antigen sensor. The sample test well can further be configured to fixably engage or couple with a base station of the system, whereby the engagement optionally puts the sample test well and the base station in mechanical and/or electronic communication. The sample test well may be in the form of a cartridge that couples with the base station or may be within a housing that electrically couples to the base station. The sample test well can optionally be single-use and disposable. The base station may be a discrete and dedicated unit to operate the antigen detection system or may be a computing device that is capable of multi-purpose computing functions, but running a computer software application that controls the antigen detection system.

In some embodiments, the homogenizing mechanism can be an impeller disposed within the well that, when turned, increases fluid flow of the sample, thereby homogenizing the sample and producing a homogenate. In further embodiments, the cartridge will include a lid configured to form a fluid-tight seal along a top side of the well. In some such embodiments, a homogenizing agent can be provided to provide a medium into which the sample can be homogenized. In yet further embodiments, the cartridge will include a drain that can direct the fluid flow of homogenate from the well into a waste receptacle.

In further embodiments, the antigen sensor may be an immunologically based sensor, such as a lateral flow immunoassay, antibody-bound piezoelectric film, or antibody-bound films used in conjunction with optical, chemical, electrical or mass sensors. Alternatively, the antigen sensor may be a mass or charge-type sensor, such as a quartz crystal microbalance that measures mass per unit area based upon changes in crystal resonance frequency upon a binding event For each of these sensor-types, an appropriate measurement indicating the presence of antigens can be used, such as a change in surface plasmon resonance, surface frequency resonance, or optical refraction index.

In some embodiments, the cartridge can include an electrophoresis system. The electrophoresis system can be configured to drive antigens towards the antigen sensor, thereby reducing testing time.

In at least one embodiment, the antigen detecting system includes a base station having a housing, the housing having cartridge dock consisting of a recessed cavity with a geometry corresponding to that of the test cartridge, such that the test cartridge may be engaged and disengaged within the recessed cavity, a sensor electrically coupled to antigen detection and analysis electronic circuitry, at least one drive motor, a power supply, and an indicator to provide either a visual or audible indicator to a user of a positive reading from the sensor. The coupling between the base station and the test cartridge may additionally serve to power on and actuate the homogenizing mechanism, the sensor and the indicator. In some embodiments, the at least drive motor may be an electric motor and axle that, when the cartridge is coupled to the base station, engages with a rotor of the homogenizing device of the test cartridge, thereby imparting rotation to the homogenization device. Alternative driving systems are contemplated by the present invention, including, for example, magnetic coupling between the drive motor and the homogenizing mechanism.

The supply may be an internal battery, preferably rechargeable, retained within the housing of the base station, an external power supply, such as that provided from an AC or DC source and electrically connected to a port within the housing, such as a Universal Serial Bus (USB) port or a DC plug receiver.

In further embodiments, the indicator will include either a single or an array of light-emitting diodes (LEDs). The LEDs optionally includes a variety of colors of LEDs, including red, yellow, and green, for example. The array of LEDs can be configured to convey a variety of messages to a user, including the on/off condition of the antigen detection system, whether a cartridge has successfully docked with the base station, if an error has been encountered during testing, the presence or absence of antigens, etc. In other embodiments, the indicator may include a liquid crystal display (LCD) capable of displaying alphanumeric or graphical characters indicating, in addition to those messages previously presented, greater detail about problems encountered during testing, the antigen density of the sample, remaining battery life, etc.

In some embodiments, the base station will include one or more communication devices capable of transmitting and receiving data to and from another electronic device in accordance with a communication standard. Examples of such communication standards include wired standards such as, for example, universal serial bus (USB), IEEE 1394 (FireWire), and IEEE 802.3 (Ethernet), and also include wireless standards such as, for example, IEEE 802.15 (Bluetooth), IEEE 802.11 (Wi-Fi), and IEEE 802.16 (WiMAX).

In accordance with another embodiment of the invention, the base station may consist of a mobile computing device, such as a tablet computer, e.g., an IPAD (Apple) or a smartphone, e.g., IPHONE (Apple) or DROID (Motorola), where the power, display and computing functions of the inventive antigen detection system are all performed by the mobile computing device. In this embodiment, a remote docking station having power and data connections to the mobile computing device is provided to electically couple to the sample test cartridge. For example, a USB cable may be employed to power the remote docking station and permit data transfer from the remote docking station to the mobile computing device. A computer software application running on the mobile computing device controls the operation and interface between the mobile computing device and the remote docking station to operate the sample test cartridge, read the antigen sensor output and display an indicator or other visual indicia to a user upon completion of a test together with the result of the test.

### BRIEF DESCRIPTION OF THE DRAWINGS

FIG. 1 is a perspective view of the antigen detection system in accordance with the present invention.

FIG. 2 is a side elevational, partial cross-sectional view taken along line 2-2 of FIG. 1.

FIG. 3 is a side elevational, partial cross-sectional view taken along line 3-3 of FIG. 1.

FIG. 4 is a top elevational fragmentary view of a base station in accordance with the present invention.

FIG. 5 is a top elevational fragmentary view of a test cartridge in accordance with the present invention.

FIG. 6 is a bottom elevational fragmentary view of the test cartridge in accordance with the present invention

FIG. 7 is a cross-sectional view taken along line 7-7 of FIG. 5.

FIG. 8 is a cross-sectional view taken along line 8-8 of FIG. 5.

FIG. 9 is a diagrammatic view representing antigen binding to immobilized antibodies within the test cartridge in accordance with the present invention.

FIG. 10 is a flow diagram depicting the method of testing for the presence of food allergens in food stuffs in accordance with the present invention.

FIG. 11 is a perspective view of an alternative embodiment of the antigen test system in accordance the present invention.

FIG. 12 is a cross-sectional diagrammatic view taken along line 12-12 of FIG. 11.

FIG. 13 is a cross-sectional diagrammatic view taken along line 13-13 of FIG. 11.

FIG. 14A is a side-elevational view of a test well cartridge and test dock in accordance with an alternative embodiment of the present invention.

FIG. 14B is an expanded view illustrating a self-assembled antibody monolayer coupled to a quartz crystal microbalance in accordance with the present invention.

FIG. 15A is a top elevational view of a test well cartridge in accordance with the present invention depicting a cover in an open position for loading a sample to be tested.

FIG. 15B is a top elevational view of a test well cartridge in accordance with the present invention depicting a cover in a closed position for loading into a test well and running a sample test.

### DETAILED DESCRIPTION OF THE PREFERRED EMBODIMENTS

The system and method of the present invention is presented with reference to the accompanying Figures. The accompanying Figures are merely for purpose of illustrating exemplary embodiments of the present invention and are not intended to limit the scope of the invention to the exemplary embodiments. Similar reference numerals among the several Figures are employed to denote identical or similar elements present in the different views presented in the Figures.

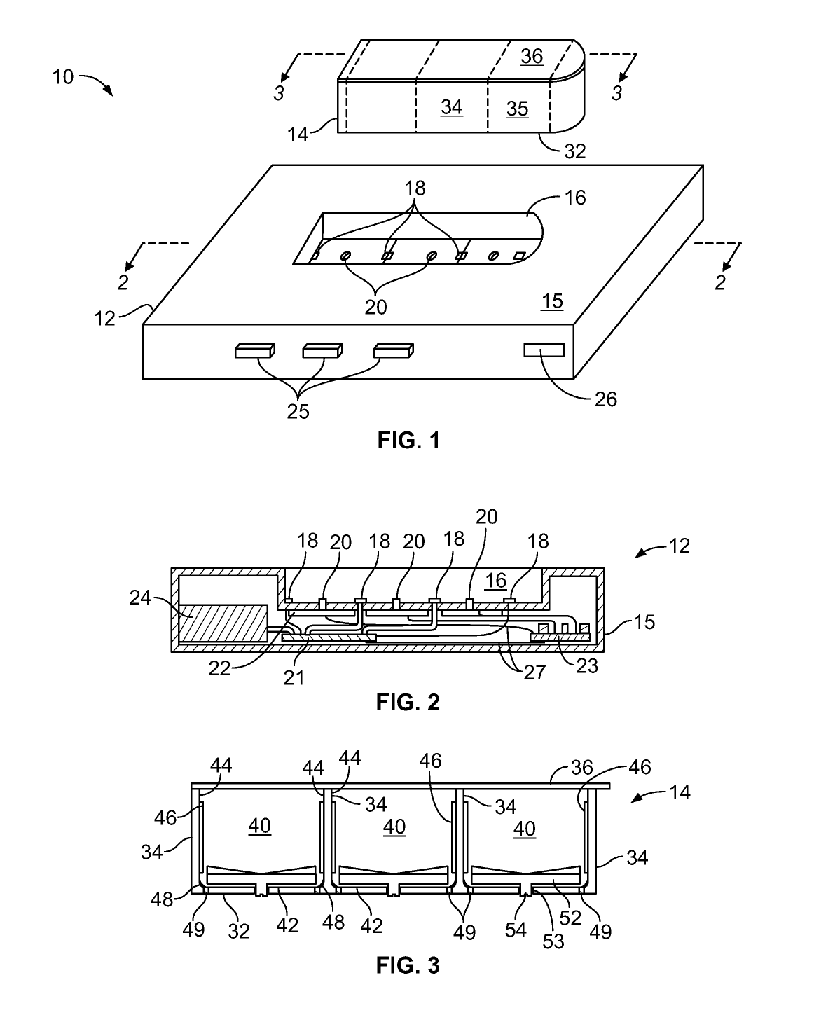

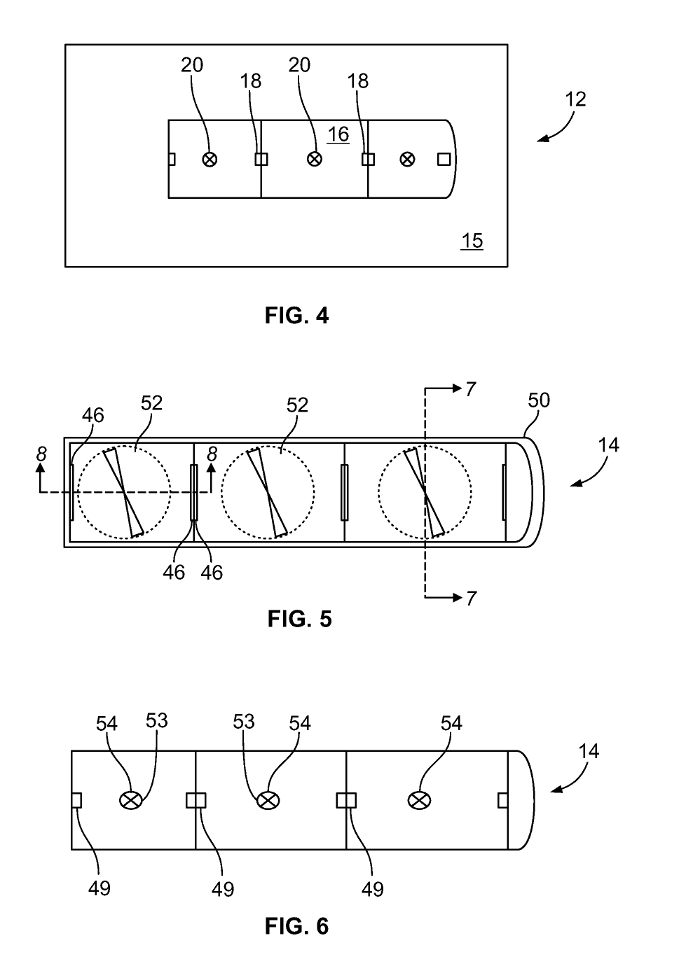

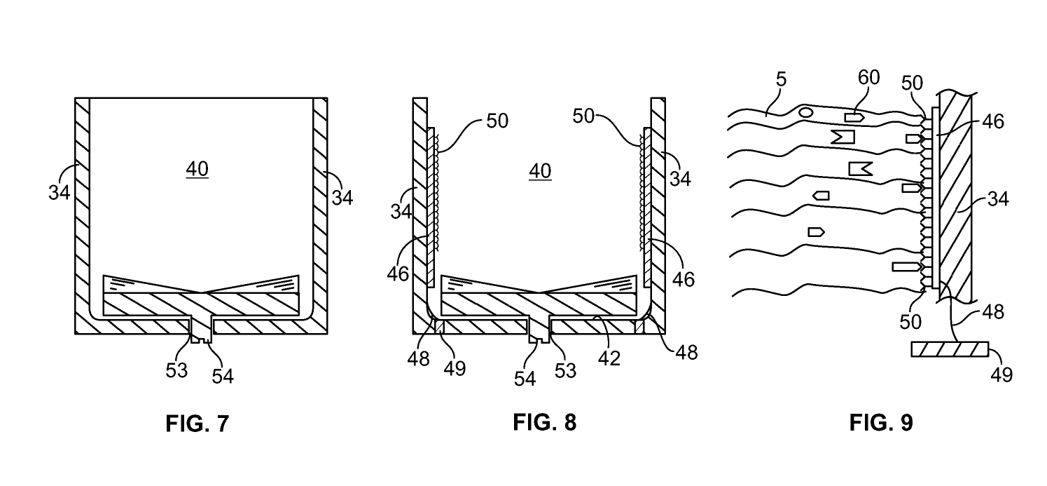

Turning now to FIGS. 1-8, the inventive food antigen detection system 10 is depicted. Food antigen detection system 10, consists generally of two components, namely, a base station 12 and a test cartridge 14. Base station 12 includes a housing 15, at least one cartridge dock 16, electrical connectors 18, at least one drive coupling 20, at least one drive motor 22, a power source 24, and an indicator 26 that produces a user detectable signal to alert a user to the presence of food allergens in a food sample being tested. Control circuitry 21 and logic circuitry 23 are provided within the housing 15. Control circuitry 21 and logic circuitry 23 electrically couple, via electrical conduits 27, to the electrical connectors 18, to the at least one drive motor 22, power source 24 and the indicator 26. It will be understood by one skilled in the art that control circuitry 21 and logic circuitry 23 may each be on dedicated or discrete circuits, such as circuit boards or integrated circuit chips or may be combined on a single circuit board or integrated circuit chip.

The test cartridge 14 includes a housing 30, the housing 30 being defined by a bottom wall 32, a plurality of side walls 34, and an openable top surface 36. Housing 30 also includes a test well 40 defined between the bottom wall 32, plurality of side walls 34 and openable top surface 36. At least some of the plurality of side walls 34 have an inner surface 44 which bounds the test well 40. A sensor 46 is embedded, coupled to or otherwise functionally associated with a portion of the inner surface 44 of the side walls 34 and exposed to the test well 40. Antibodies 50 are immobilized onto the sensor 46 such that they are exposed to the test well 40 for binding to antigens. Electrical conduits 48 couple sensor 46 to electrical contacts 49 on either an outer bottom surface 52 of bottom wall 32 or an outer side wall surface 54 of side wall 34.

Control circuitry 21 governs the electrical current supplied to and received from the at least one drive motor and the electrical connectors 18, and, therefore the test cartridge 10. Logic circuitry 23 determines a difference in electrical signal between that supplied and that returned from the sensor 46. If that difference exceeds a predetermined threshold signal difference and the logic circuitry 23 activates indicator 26.

The bottom wall 32 has an inner bottom surface 42 bounding the test well 40. A homogenizing element 52 is disposed with test well 40 in order to mix food samples placed within the test well 40. The homogenizing element 52 may be a rotating vaned member, such as that depicted in FIGS. 3 and 5, a magnetic stirring rod, beads, or other structure suitable for macerating and mixing food sample placed within the test well 40. It is preferable that homogenizing element 52 be positioned within the test well adjacent to the inner bottom surface 42 to permit coupling of the homogenizing element to the drive motor 22 in the cartridge dock 16 of the based station 12. Where, as illustrated in FIGS. 3 and 5, the homogenizing element 52 is a rotating vaned member, the bottom wall 32 of the test cartridge 14 may have an opening 53 that communicates between the inner bottom surface 42 and the outer bottom surface 52 and permits a coupling member 54 to pass through the bottom wall 32 of the test cartridge 14. The coupling member 54 may be a mechanical linkage, such as a clutch or friction disk, or may be a magnetic coupling. In determining the type of engagement between the homogenizing element 52 and the drive motor 22, it is important to consider the rotational speed and torque that needs to be applied to the homogenizing element 52 in order to macerate and mix the food sample being tested. Highly fibrous or viscous foodstuffs may require different speeds and torques than looser or lesser fibrous foodstuffs will require in order to obtain a degree of maceration and mixing necessary to expose antigens within the foodstuffs and allow for antibody binding, sensor detection and activation of the indicator, all within 30-60 seconds of activation of the homogenizing element 53.

It will be understood that test cartridge 14, while depicted with three wells 40, may be configured with a single test well 40 or with different numbers of test wells 40, such as two, four, five, etc. depending upon the specific design desired. Where multiple test wells 40 are employed, it may be preferable to allow the user to select which test well 40 to select for testing. Thus, switches 25 may be provided in the housing 15 of the base station, to permit the user to select a corresponding test well 40 to activate and test. In the embodiment depicted in FIG. 1, three test wells 40 are depicted and three switches 25 are provided, with one switch 25 activating a single test well. Alternatively, a single switch 25 may be employed to select between multiple test wells 40.

Sensor 46 may be any type of device capable of generating a detectable electronic signal upon meeting threshold of antigen binding to antibodies 50 immobilized on the sensor 46. The sensor 46 may be a lateral flow immunoassay, piezoelectric, or optical sensors. With each type of sensor, antibodies 50 specific for a given food antigen, e.g., nuts, shellfish, gluten, etc., are immobilized onto the sensor 46 and a cause a change in surface plasmon resonance, surface frequency resonance, or optical refraction index of the sensor 49, which, in turn, generates an electrical signal indicative of the change and activates the indicator 26.

In accordance with one preferred embodiment of the present invention sensor 46 is a piezoelectric sensor. The piezoelectric sensor is associated with a surface of the test well 40, such as the inner side wall 44 or the inner bottom surface 42, such as by adhering, molding, recessing, mechanically attaching or other such equivalent way of coupling the sensor to the test well 40. Electrical conduits 48 are coupled to the sensor 46 and communicate with external connectors 49 in or on the test cartridge housing 30. The external connectors 49 serve to electrically couple the test cartridge 14 to the base station 12 through the electrical connectors 18 in the cartridge dock 16. The external connectors 49 and the electrical connectors 18 preferably align with one another when the test cartridge 14 is engaged with the cartridge dock 16 of the base station 12. The electrical connectors 18 and the external connectors 49 may be configured simply as contacts, may be plug and spade type connectors, pin and recess connectors or other suitable configurations for establishing electrical communication there between.

Antibodies 50 may be immobilized on the sensor 46 by various means that are well known in the biotechnology arts. Crosslinking and adsorption methods for binding antibodies to substrates are disclosed in U.S. Pat. No. 7,329,536 to Zeng, et al., which is hereby incorporated by reference. Alternatively, gel-based immobilization agents may be employed and then coated onto a substrate surface as disclosed in U.S. Pat. No. 6,696,264 to Bodenhamer, W., et al, which is also incorporated by reference. It will be understood that the specific means for binding or immobilizing the antibodies 50 to the sensor 46 is considered within the purview of one of ordinary skill in the art.

Non-limiting examples of antibodies and antigens suitable for use with the present invention are the following: Anti-AraHl (peanut), rabbit-sourced polyclonal antibodies, Anti-AraH2 (peanut), rabbit-sourced polyclonal antibodies, natural sourced AraH1, AraH2, AraH6 antigens from peanut, natural sourced ovomucoid, albumin, and Gal GD4 antigens from egg, and natural sourced tropomycin antigen from shrimp. Alternately, one skilled in the art may use established methods of creating polyclonal animal (e.g., goat, rabbit, sheep) serum sourced antibodies as well as monoclonal antibodies using hybridomas.

FIG. 9 is a diagrammatic view of a sensor 46 having bound antibodies 50 on its surface, with antigens 60 present in a food sample 5 binding to the antibodies 50.

As noted above, an alternative embodiment of the invention may include an anode and cathode in each of the test wells 40 in order to permit electrophoretic separation of the antigens from the food sample. Where electrophoresis is employed as an adjunct, reagents suitable for electrophoresis may be added to the test well 40, either in wet or dry form, to enhance antigen extraction, food homogenization, as well as augment reaction rate of detection. Such types of reagents may, optionally, be used in non-electrophoresis configurations of the present invention.

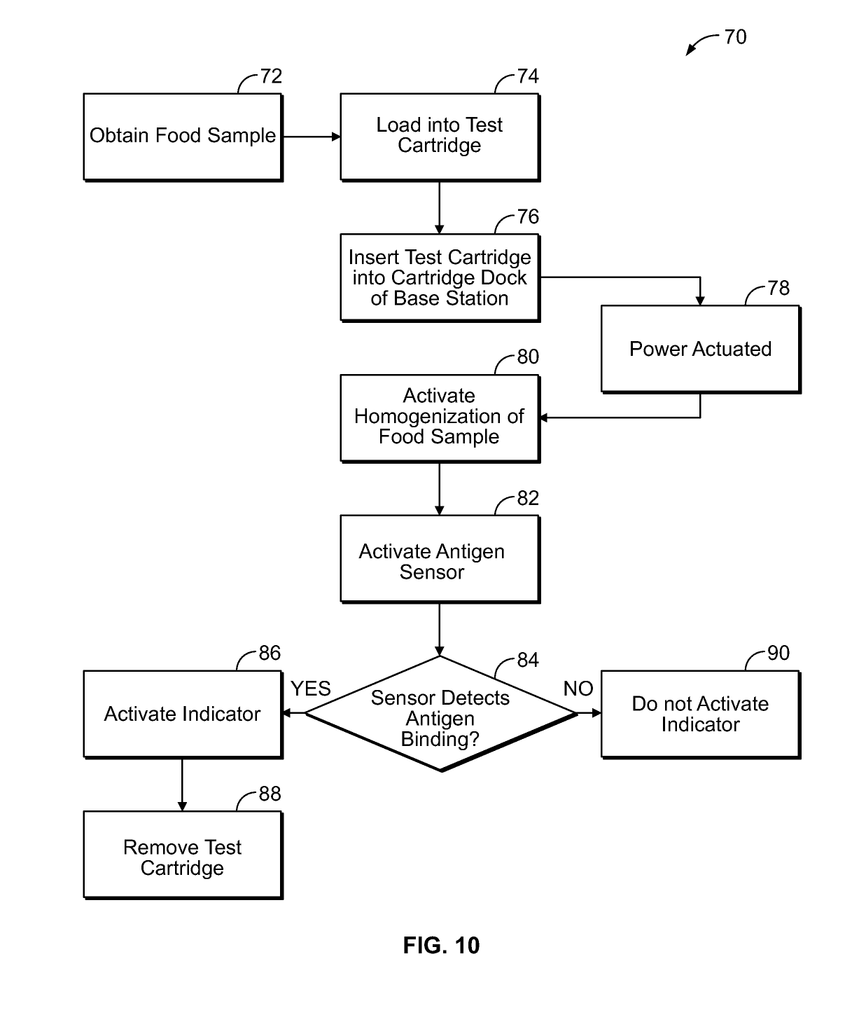

FIG. 10 is a flow diagram illustrating the method 70 of detecting the presence of food-borne antigens in accordance with the present invention. In accordance with method 70, a food sample is first obtained 72 and loaded 74 into a test well 40 of the test cartridge. The user then inserts the test cartridge into the cartridge dock of the base station at step 76, which actuates power to the test cartridge 78 and activates the maceration and homogenization of the food sample within the test well 40 at step 80. Concurrently the antigen sensor is powered and activated at step 82. A logical step 84 determines if a given threshold of antigen binding is detected. If so, the indicator is activated 86 and the user may remove the test cartridge 88. If a threshold level of antigen binding is not detected the indicator is not activated 90 and the user may either remove the test cartridge or retest another food sample.

### EXAMPLE 1

One liter of chicken broth is brought to a boil. One ml of peanut oil is added to the boiling broth and the mixture is allowed to simmer covered for 30 minutes. A test cartridge as described above, is provided, with Anti-AraH1 (peanut), rabbit-sourced polyclonal antibody [Indoor Biotechnology, Inc., Charlottesville, Va.) are immobilized in an agarose gel and applied to a piezoelectric film adhered to wall surfaces of a test well. 5 ml of the soup and peanut oil mixture is introduced into the test well and the test cartridge is inserted into the cartridge dock of the above-described base station. Upon engaging the test cartridge into the cartridge dock, 5 mA of DC current is applied to the piezoelectric film to establish a resonance frequency and, concurrently, 9V DC is applied to rotate the homogenizing element within the test well. After 60 seconds, a visual indicator illuminated indicative of the presence of antigen bound antibodies on the piezoelectric sensor.

### EXAMPLE 2

The conditions of Example 1 are repeated, except that 25 grams of stewed carrots, 300 grams of dry wheat noodles, 5 mg salt, 5 mg pepper and 0.1 ml of peanut oil were added to 2 liters of chicken broth before bringing it to a boil. After simmering for 30 minutes 5 ml of the soup was introduced into the test well and the test cartridge inserted into the cartridge dock for 60 seconds. After 60 seconds, no illumination of the visual indicator was noted indicating a sub-threshold level of antigen binding to the antibodies.

### EXAMPLE 3

An additional 1.9 ml of peanut oil were added to the soup mixture prepared in Example 2, the soup was again brought to a boil and simmered for 30 minutes. 5 ml of the soup was introduced into the same test well used in Example 2 and the test cartridge reinserted into the cartridge dock for 60 seconds. After 60 seconds the visual indicator illuminated indicating the presence of threshold binding of the antigen to the antibodies on the piezoelectric sensor.

### EXAMPLE 4

Three test and three control quartz crystal microbalance (QCM) was prepared with 10 ul of a peanut antibody (AB) (polyclonal rabbit anti-Ara h1, Indoor Biotechnologies) tethered in a self-assembled monolayer to the quartz crystal microbalance. After drying for two hours at room temperature, the frequency of each crystal (f0) was tested and recorded. Three dilutions of peanut antigen (AG) (Indoor Biotechnologies) were prepared for testing at 100:1, 200:1 and 400:1 dilution ratios, 10 ul of each antigen dilution were pipetted onto each test QCM. 10 ul of of deionized water (DI) was added to each of the control QCMs. The change in frequency between f0 and the initial motional resistance (r0) of the crystal and the test motional resistance (rf) were measured each minute between 0 minutes (time of introduction of the antigen) to 2 minutes and the frequency shift noted. Table 2, below, summarizes the recorded frequency shift data and demonstrates that a significant detectable shift in frequency is detected and detectable even at 400:1 dilutions of AG relative to the control DI.

TABLE 2

| | 100:1 AB | | | | 200:1 AB | | | | 400:1 AB | | | |
|---|---|---|---|---|---|---|---|---|---|---|---|---|
| | Dilution | | | | Dilution | | | | Dilution | | | |
| f0 | 4975954 | Hz | 4973078 | Hz | 4986745 | Hz | 4975333 | Hz | 4978293 | Hz | 4983949 | Hz |
| r0 | 36.7 | Ohms | 12.8 | Ohms | 12.2 | Ohms | 10.6 | Ohms | 10.5 | Ohms | 7.5 | Ohms |
| rf | 148.0 | Ohms | 171.0 | Ohms | 163.7 | Ohms | 179.5 | Ohms | 176.3 | Ohms | 153.6 | Ohms |

| | AG* | DI** | AG-DI*** | AG* | DI** | AG-DI*** | AG* | DI** | AG-DI*** |
|---|---|---|---|---|---|---|---|---|---|
| | delta | delta | delta | delta | delta | delta | delta | delta | delta |
| | frequency | frequency | frequency | frequency | frequency | frequency | frequency | frequency | frequency |
| | (Hz) | (Hz) | (Hz) | (Hz) | (Hz) | (Hz) | (Hz) | (Hz) | (Hz) |
| 0 min | 1420 | 1130 | 290 | 536 | 375 | 161 | 136 | 39 | 97 |
| 1 min | 1390 | 1180 | 210 | 515 | 360 | 155 | 135 | 42 | 93 |
| 2 min | 1377 | 1152 | 225 | 514 | 356 | 158 | 139 | 41 | 98 |

Finally, in accordance with an alternative embodiment of the present invention, the base station may include one or more communications circuitry capable of transmitting and receiving data to and from another electronic device in accordance with a communication standard. Examples of such communication standards include wired standards such as, for example, universal serial bus (USB), IEEE 1394 (FireWire), and IEEE 802.3 (Ethernet), and also include wireless standards such as, for example, IEEE 802.15 (Bluetooth), IEEE 802.11 (Wi-Fi), and IEEE 802.16 (WiMAX). Moreover, the communications circuitry may include Global Positioning Satellite (GPS) which tracks and can record to digital memory, the user's location when a test is conducted. When operated in conjunction with publicly available mapping databases, such as GOOGLE EARTH, GOOGLE MAPS or MAPQUEST have the capability to look up and record the particular restaurant name, address, telephone number, website Uniform Resource Locator (URL) and link to other information, such as reviews about the restaurant. In this manner the inventive antigen detection system 10 may be used to not only test food samples for a user, but to provide a data source for interactive communications with social media networks existing on the Internet in order to provide others with information concerning the presence or absence of food allergens in prepared food products, such as at restaurants.

Additionally, by providing the food allergen detection system 10 with communications capability, bidirection communications between the system 10 and remote computers, such as over the Internet, to a laptop computer, to a desktop computer, to a smartphone, such as an IPHONE, ANDROID or BLACKBERRY or to a tablet computer, such as an IPAD, all permit connectivity for a wide variety of purposes such as those for which other computing or communications devices are employed. For example, the system 10 may upload or receive data from the Internet, communicate with websites, allow for remote diagnostics of the operation of system 10, permit software or firmware updates to be handled through remote connections, allow for system diagnostics using software loaded on a remote computing device or one to which the system 10 is connected locally.

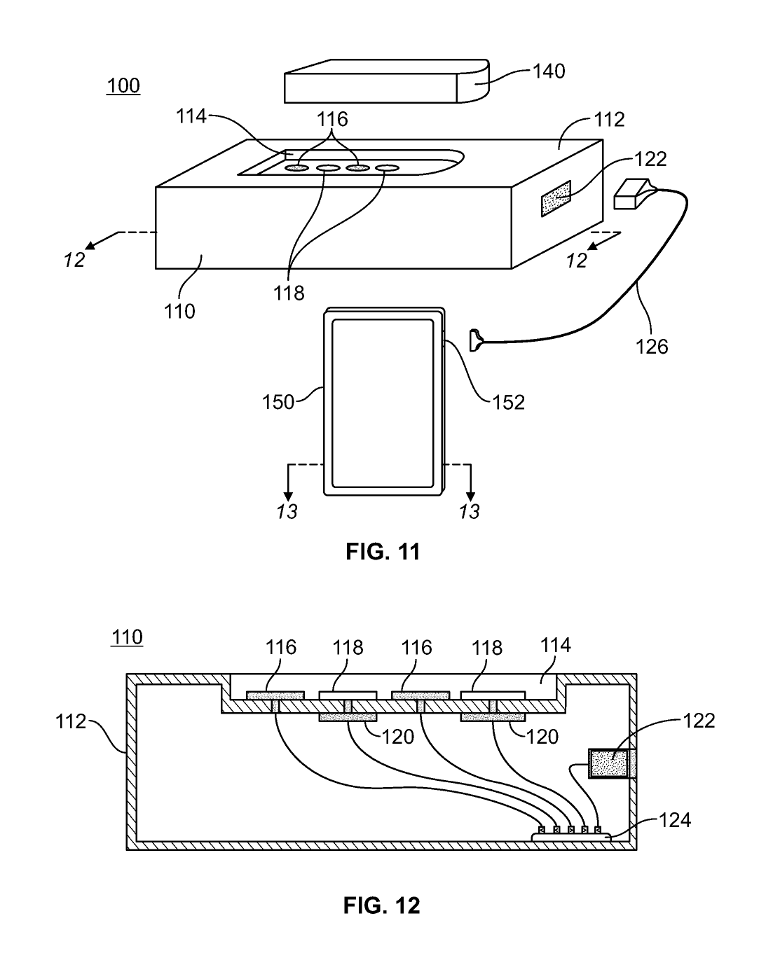

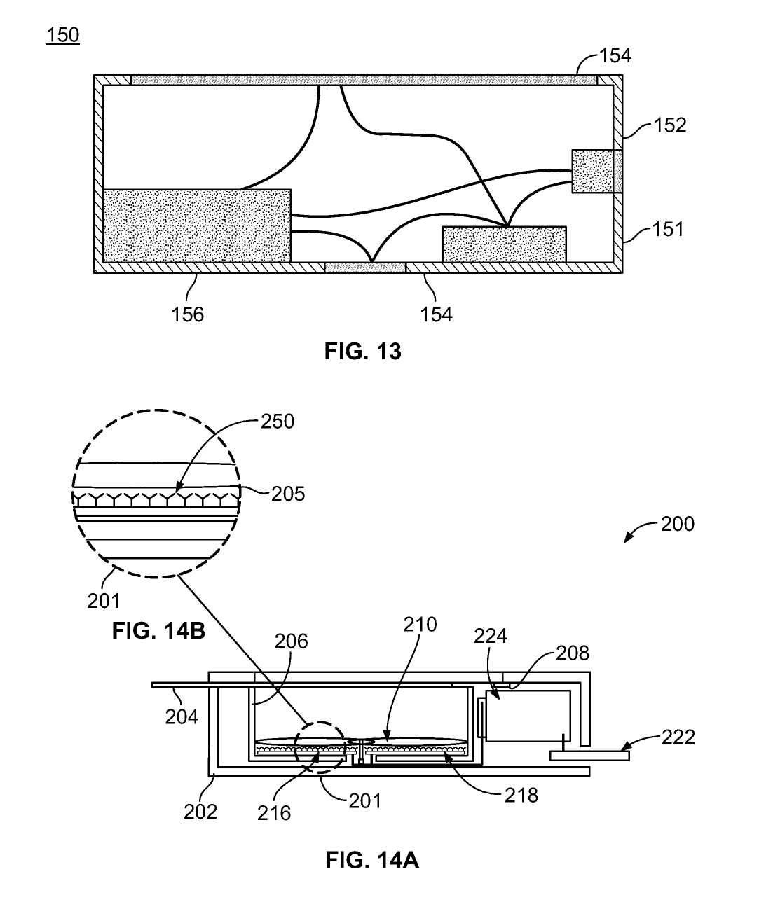

In a further embodiment, as depicted in FIGS. 11-15, a food allergen detection system 100 comprises a base station 110 and a test cartridge 140. The base station 110 includes a housing 112, at least one cartridge dock 114, electrical connectors 116, at least one drive coupling 118, at least one drive motor 120, an electronic port 122, and circuitry 124. The circuitry 124 is coupled electronically to the at least one drive motor 120 and the electronic port 122. The electronic port 122 is configured to couple with a wired cable 126 such that the circuitry 124 can both send and receive data via the wired cable 126 and such that electricity can be transferred to the food allergen detection system 100 via the wired cable 126. The test cartridge 140 is essentially as described above and shown in FIG. 2, including a sensor, electrical conduits, electrical contacts, and a homogenizing element.

In the present embodiment, the function provided by the control and logic circuitry in previous embodiments is performed by an external computer 150, such as a smartphone, tablet computer, or personal computer, which typically includes a microprocessor 151. The external computer 150 includes a connection port 152 that conforms to any wired industry standard, such as universal serial bus (USB), IEEE 1394 (FireWire), PCI, PCI Express, Thunderbolt, and IPHONE serial dock connector.

The electronic port 122 may be configured to comply with the physical and electrical communication standards in the industry, such as USB, FireWire, PCI, PCI Express or the IPHONE serial dock connector. The food allergen detection system 100 can receive instructions from the external computer. The instructions received can include instructions to control operation of the drive motor 120, thereby actuating the homogenizing element within the test cartridge 140. The instructions received can further control operation of the sensor via the circuitry 124, electrical contacts 116, and the electrical conduits to detect the presence of food-borne antigens as described above. When the sensor 142 is in operation, the resulting signal is delivered to the external computer 150 via the wired cable 126. It will be appreciated by those skilled in the art, that the resulting data signal may also be transmitted from sensor 142 to the external computer 150 wirelessly.

The external computer 150 determines a difference in electrical signal between that supplied and that returned from the sensor 142. For example, the microprocessor 151 may issue a command to the sensor 142 that, in turn, generates a first signal representing a first measurement. That first measurement is then returned to the microprocessor 151. The sensor 142 then generates a second signal representing a second measurement that is also sent to the microprocessor 151. A change from the first measurement to the second measurement above a predetermined threshold indicates the presence of the target antigen.

The presence or absence of the target antigen is communicated to a user via an indicator 154. The method of indication used by the indicator 154 depends on the capabilities of the external computer 150, but includes audio notification such as a tonal indication or a spoken indication, and also includes visual notification such as a pictorial and video indication or a textual indication, and combinations thereof. These methods of indication may be accomplished by technological devices known in the industry, including visual displays and loudspeakers.

In the present embodiment, the electricity necessary to operate the various electrical elements of the food allergen detection system 100 is provided via the wired cable 126. The external computer 150 includes a power source 156, for example, a battery or a connection to a power grid. In each of the above mentioned communication standards, electricity is delivered through the attending cable. The circuitry 124 includes circuitry capable of manipulating and/or conditioning the electricity provided by the wired cable 126 to conform to the requirements of the various electrical components of the food allergen detection system 100 and delivering the electricity thereto.

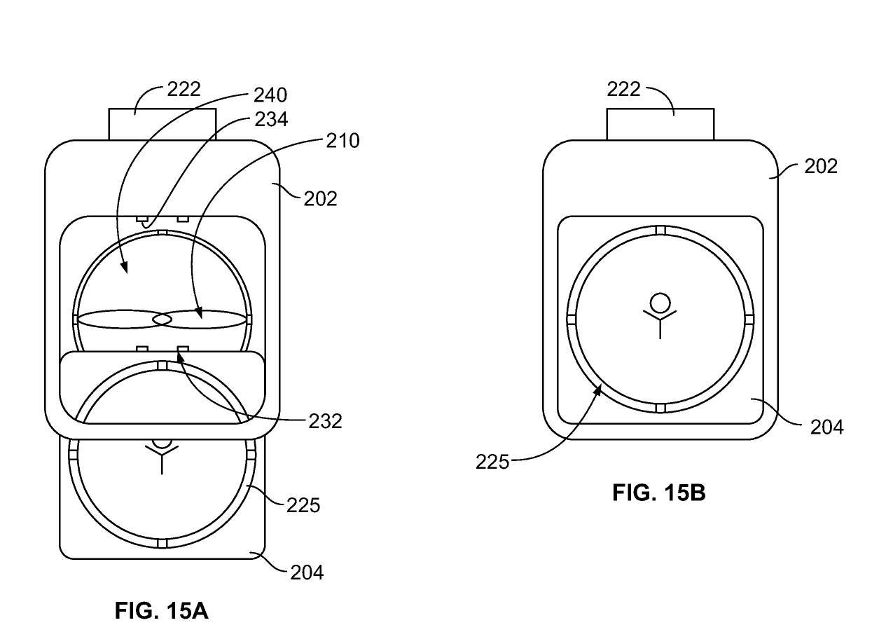

FIGS. 14A and 14B and 15A and 15B depict another embodiment of the sample test cartridge 200 in accordance with the present invention. Sample test cartridge 200, like cartridge 100 depicted in FIG. 11, is configured for remote electrical connection to a base station, such as external computer 150. Sample test cartridge 200 consists generally of an external housing 202 defining an inner sample test well 206 and housing an electrical connector 222, such as a USB interface connector, a circuit board 224, an impeller or homogenizing element 210 positioned in the inner sample test well 206 and an antigen sensor consisting of mass transport sensor, such as a quartz crystal microbalance (QCM) having a control section 206 and a test section 210. Antibodies 250 are bound to both the control section 206 and the test section 210 of the mass transport sensor, such as the QCM, specific to the particular antigen to be detected. In accordance with one embodiment of the invention, the antibodies 250 are formed as a self-assembling tethered antibody monolayer bound to the mass transport sensor, such as the QCM. The impeller or homogenizing element 210 is electrically connected to a drive element that, in turn is connected to the circuit board 224. Like with the above-described embodiments, antigen present in the sample 205 will bind specifically to the antibodies causing a signal change in the mass transport sensor that may be detected using the external computer 150.

As depicted specifically in FIGS. 15A and 15B, the sample test cartridge 200 may include a sliding cover 204 that both covers the test well 240 after a sample is introduced, and includes the visual indicator 225, such as LED lights, to provide the user with a positive visual signal indicative of the positive or negative results of a test run. In this embodiment, the visual indicator 225 is powered by the electrical coupling of electrical contacts 232 in the sliding cover 204 and electrical contacts 234 in the cartridge housing 202, which are, in turn, electrically coupled to the circuit board 224 and electrical connector 222. The entire sample test cartridge

In a further embodiment, the invention can detect other antigens capable of being immobilized by surface-bound antibodies. For example, tumor antigens, either tumor-specific antigens or tumor-associated antigens, can be detected by taking a biological sample from a patient and depositing the biological sample within a test cartridge as described above. Tumor antigen-specific antibodies are bound to the test surfaces and detect the presence of said tumor antigens in the same method as described above. Examples of other biological samples within the scope of the invention include, but are not limited to, saliva, urine, fecal matter, blood, biopsied matter, secreted substances, and all other biological matter known in the art. In another example, the antibodies bound to the test surface can target an antigen found in a non-biological sample. Such non-biological samples include environmental samples, including water, soil, and plant matter. Target antigens within the samples include tumor indicators, pathogens, such as bacteria, viruses, protozoa, fungi, prions, and parasites.

In a further embodiment, the invention can detect the presence of antibodies in a sample. In this embodiment, antigens configured to target a specific antibody are bound to the test surfaces of the test cartridge. When a biological sample containing the target antibody is deposited within the test cartridge and homogenized as described above, the binding of the antibodies to the surface-bound antigens causes a detectable change in the test surface as described above. Examples of antibodies included within the invention are IgA, IgD, IgE, IgG, and IgM.

Those skilled in the art will understand and appreciate that while the present invention has been described with reference to exemplary embodiments, such embodiments are not intended to restrict the intended scope of the invention, which is limited only by the claims appended hereto. It will be further appreciated that variations in antigen, antibody, binding or immobilization technique or reagents, food stuffs being tested, or the like may be made without departing from the intended scope of the invention.

1. A detection system for detecting antigens in foodstuffs, comprising:
a) a sample test well having an antigen detection sensor disposed within the sample test well;
b) antibodies specific for a predetermined food antigen, bound to the antigen detection sensor;
c) a computer controller electrically connected to the antigen detection sensor and the sample test well; and
d) an indicator coupled to the computer controller for indicating the presence of absence of the predetermined food antigen in a foodstuff sample.

2. The detection system of claim 1, wherein the electrical connection between the computer controller and the antigen detection sensor and sample test well is selected from the group of universal serial bus, IEEE 1394, PCI, PCI Express, Thunderbolt, BLUETOOTH and IPHONE serial dock.

3. The detection system of claim 1, wherein the computer controller supplies electrical power to the sample test well and antigen detection sensor.

4. The detection system of claim 1, wherein the computer controller is selected from the group consisting of smartphones, tablet computers, and personal computers.

5. The detection system of claim 1, wherein the indicator includes at least one of a visual display and an audible signal generator.

6. The detection system of claim 1, further comprises a macerator disposed within the test well to homogenize the foodstuff sample within the sample test well.

7. The detection system of claim 1, wherein the sample test well further comprises a chamber within a cartridge housing and the computer controller further comprises a base station having a computer controller circuit board and a mating dock for operably coupling the cartridge to the base station.

8. A method of detecting antigens in a sample of foodstuff, comprising the steps of:
a) disposing a foodstuff sample to be tested within a sample testing well;
b) macerating the foodstuff sample to be tested within the sample testing well;
c) binding foodstuff antigen to antibodies bound on an antigen detection sensor disposed within the sample testing well;
d) detecting the presence or absence of antigen bound antibodies at the antigen detection sensor;
e) generating a signal indicative of the binding or non-binding of the antigen to the antibodies at the antigen detection sensor; and
f) generating at least one of a visual or audible signal indicative of the binding or non-binding of the antigen to the antibodies at the antigen detection sensor.

9. The method of claim 8, further comprising the step of adding a homogenizing agent within the sample test well as part of the macerating step.

10. The method of claim 9, further comprising the step of disposing a lid on top of the sample test well thereby forming a fluid tight seal.

11. The method of claim 8, wherein steps a-e are carried out in a sample test well housing member and wherein step f is carried out by a computing device electrically coupled to and discrete from the housing member.

12. The method of claim 8, wherein the antigen detection sensor senses binding of antigen to antibodies by at least one detection method selected from the group consisting of resonance frequency, surface plasmon resonance frequency, mass detection, chemical detection and optical detection.

13. The method of claim 8, wherein the step of indicating the presence or absence of the target antigen uses at least one indication method selected from the group consisting of tonal indication, spoken indication, pictorial indication, and video indication.

14. The method of claim 11, wherein the computer provides electrical power to the sample test well housing member.

15. A detection system for detecting antibodies in a sample, comprising:
a) a base station comprising a housing, a cartridge dock, a rotary drive motor, and an electronic port;
b) a cartridge removably engageable with the cartridge dock, the cartridge defining a well, an impeller disposed within the well and capable of operably coupling to the rotary drive motor, antigens specific for a predetermined antibody disposed on one or more wall surface of the well, and a sensor operably coupled to the antibodies and the electronic port;
c) a computer having a microprocessor, a connection port, a power supply, and an indicator; and
d) a wired connector electronically coupled to the electronic port and the connection port.

16. The detection system of claim 15, wherein the antigens specific for a predetermined antibody target at least one of the antibodies selected from the group consisting of IgA, IgD, IgE, IgG, and IgM.
